(19) United States
(12) Patent Application Publication
(10) Pub. No.: US 2003/0134906 A1
(43) Pub. Date: Jul. 17, 2003

(54) Modified release pharmaceutical composition containing bupropion HCI as active substance

(75) Inventors: Roberto Valducci, Savignano sul Rubicone (IT); Tiziano Alighieri, Rimini (IT); Serozh Avanessian, Rimini (IT)

Correspondence Address: William J. Sapone, 714 Colorado Avenue, Bridgeport, CT, 06605, US

(73) Assignee: VALPHARMA S.A., Serravalle (SM)

(21) Appl. No.: 10/159,184

(22) Filed: May 31, 2002

(30) Foreign Application Priority Data
Jul. 9, 2001 (IT) MI2001A 001457

(51) Int. Cl. A61K 31/137; A61K 9/22

(52) U.S. Cl. 514/651; 424/468

(57) ABSTRACT

Pharmaceutical composition in modified release tablet form containing Bupropion HCl as active substance and including, at the same time, hydrophilic components and hydrophobic ingredients mixed with an excipient substance.

### STATE OF THE ART

Bupropion HCl is a substance having interesting pharmacological characteristics, similar to those of the tricyclic antidepressants.

Nevertheless Bupropion HCl has an elevated hygroscopicity and susceptibility to the decomposition.

For this reason various compositions with stabilizing intent have been studied.

For example, the patents n° WO 95/03781 and U.S. Pat. No. 5,541,231 describe compositions in solid form in which the presence of various acid substances gives stability to Bupropion HCl.

The patents n° U.S. Pat. No. 5,427,798 and EP 656775 describe controlled release tablets obtained through the technique of hydrophile matrixes based on hydroxypropylmethylcellulose.

However, the stability and the dissolution profile are not satisfactory.

Further modified release compositions are described in the patent n° EP 0171457, which provides for the preparation of a Bupropion HCl core with osmotic components, and then the coating of said core with a membrane insoluble in water but permeable. In such membrane, soluble water substances are suspended, whose solubilization allows to “perforate” the membrane thus enabling the release of Bupropion HCl.

These techniques have the disadvantage of being very complicated and laborious.

### SUMMARY

Now we have found pharmaceutical compositions containing Bupropion HCl as active substance, which allow to overcome the disadvantages of the prior art.

Such compositions are in tablet form and are characterized by the simultaneous presence of hydrophilic substances and hydrophobic substances.

In particular, the compositions according to this invention include hydroxypropylmethylcellulose, or polyethylene oxide, stearic acid and carnauba wax and an excipient substance like lactose.

Using the various ingredients in the proper proportions it is possible to modulate the dissolution profile of Bupropion HCl as requested by the European Pharmacopoeia and to obtain tablets suitable for all the commonly utilized dosages.

Furthermore the compositions according to this invention show an elevated reproducibility of the chemical-physical characteristics and the dissolution profile.

### DESCRIPTION OF THE INVENTION

The characteristics and the advantages of the pharmaceutical compositions containing Bupropion HCl as active substance according to this invention will be better explained through the following detailed description and through the examples of preparation and characterization.

Such compositions are prepared in tablet form and include hydrophilic components and hydrophobic components.

A preferred preparation includes, besides Bupropion HCl (BP), hydroxypropylmethylcellulose (HPMC), stearic acid (SA) and carnauba wax (W), as well as an excipient substance like lactose (L). In another preferred preparation hydroxypropylmethylcellulose is replaced by polyethylene oxide. For the tablet preparation, the various components in powder form are accurately mixed. The obtained mixture is extruded at a temperature ranging from 45° C. to 65° C. to obtain a granulate.

The granulate is mixed with lubricating substances commonly utilized in the pharmaceutical technique and then transformed into tablets.

Each tablet has a Bupropion HCl content ranging from 50 mg to 500 mg.

In the composition preparation according to this invention the various components are used in the following weight proportions:

BP/(HPMC+SA+W) from 0.5 to 2;

HPMC/SA/W from 1/1/1to 1/4/4;

BP/L from 1 to 2.

As stated above it comes out that the preparation process has the advantage of being realized through more simple operations in comparison with the prior art.

Furthermore it allows the obtainment of a stable, not hygroscopic, modified release composition, having reproducible characteristics and suitable for all the commonly used dosages.

For a better explanation of the invention the following examples are reported.

### EXAMPLE 1

In a Viani granulator, type ST 25, the following ingredients in powder form were mixed:

| | |
|---|---|
| Bupropion HCl | 9.000 g |
| Hydroxypropylmethylcellulose K15 | 1.800 g |
| Lactose | 5.900 g |
| Stearic Acid | 1.800 g |
| Carnauba Wax | 1.800 g |

The obtained mixture was extruded through a Kahl extruder, model press 14-175, with a 0.8 mm net, maintaining the granulation temperature at 50° C. The so obtained granulate was mixed with lubricating substances (magnesium stearate and anhydrous colloidal silica in quantity of 2 and 1 mg per tablet, respectively) and, therefore, compressed into tablets. Each tablet having the average weight of 300 mg had a Bupropion HCl content of 150 mg. The tablets were characterized by the Bupropion HCl release utilizing the method of the European Pharmacopoeia (Paddle apparatus) and the following results were obtained:

Percentage release

| 1 h | 2 h | 4 h | 8 h | 12 h |
|---|---|---|---|---|
| 30 | 45 | 67 | 94 | 100 |

The above indicated tablets were coated and coloured to improve their appearance and protection: such coating leaves the dissolution characteristics unchanged.

### EXAMPLE 2

With the same method described in example n. 1 a granulate with the following composition was prepared:

| | |
|---|---|
| Bupropion HCl | 590 g |
| Hydroxypropylmethylcellulose K100 | 96 g |
| Hydroxypropylmethylcellulose K4 | 100 g |
| Lactose | 648 g |
| Stearic Acid | 196 g |
| Carnauba Wax | 196 g |

With a portion of the granulate, tablets containing 100 mg of Bupropion HCl were prepared.

Utilizing the same method of the preceding example the following results could be obtained:

Percentage release

| 1 h | 2 h | 4 h | 8 h | 12 h |
|---|---|---|---|---|
| 34 | 51 | 72 | 96 | 103 |

With the remaining granulate, tablets containing 150 mg of active ingredient were prepared; the so obtained tablets have been coated and coloured. In the in vitro release evaluation the following results were obtained:

Percentage release

| 1 h | 2 h | 4 h | 8 h | 12 h |
|---|---|---|---|---|
| 30 | 43 | 62 | 85 | 101 |

### EXAMPLE 3

In a Z double jacket mixer granulator, warmed at 60° C., LLEAL model AM-5, granules with the following composition were prepared:

| | |
|---|---|
| Bupropion HCl | 300 g |
| Hydroxypropylmethylcellulose K100 | 100 g |
| Lactose | 200 g |
| Stearic Acid | 200 g |
| Carnauba Wax | 200 g |

The granules were forced through a net with 1000 micrometers aperture size. The so sieved granules were mixed with lubricants and transformed into tablets containing 100 mg of active ingredient: the tablets were analysed obtaining the following results:

Percentage release

| 1 h | 2 h | 4 h | 8 h | 12 h |
|---|---|---|---|---|
| 33 | 48 | 70 | 84 | 94 |

### EXAMPLE 4

### Comparison

Example n. 3 was repeated, with the difference that in the preparation of the composition hydroxypropylmethylcellulose was not included and, therefore, the example was carried out without the hydrophilic component.

The tablets were analysed obtaining the following results:

Percentage release

| 1 h | 2 h | 4 h | 6 h | 8 h | 12 h | 16 h | 20 h |
|---|---|---|---|---|---|---|---|
| 23.0 | 31.4 | 42.5 | 50.4 | 56.5 | 65.6 | 72.1 | 76.7 |

The active ingredient dissolution patterns turned out to be very slow and not capable of being modulated.

### EXAMPLE 5

Tablets with the same procedure and composition of example n. 3 were prepared, replacing Hydroxypropylmethylcellulose with Polyethylene Oxide having a molecular weight of 1.000.000; the analysed tablets gave the following results:

Percentage release

| 1 h | 2 h | 4 h | 8 h |
|---|---|---|---|
| 39 | 62 | 84 | 100.5 |

### EXAMPLE 6

In order to obtain a three layer tablet formulation the following granulates were prepared:

6.1 Bupropion HCl was mixed with the excipients in powder, wet with the PVP solution and forced in a net having 1000 micrometers aperture size:

| | |
|---|---|
| Bupropion HCl | 1.000 g |
| Lactose | 400 g |
| Microcrystalline Cellulose | 200 g |
| PVP at 20% in Ethanol | 320 g |

The granules were transferred into a desiccator and desiccated for 24 hours at 40° C.

6.2 Utilizing the method described in example n. 3, granules with the following composition were prepared:

| | |
|---|---|
| Bupropion HCl | 300 g |
| Hydroxypropylmethylcellulose K100 | 50 g |
| Lactose | 180 g |
| Stearic Acid | 180 g |
| Carnauba Wax | 180 g |

6.3 Utilizing the method described in example n. 3, granules with the following composition were prepared:

| | |
|---|---|
| Bupropion HCl | 300 g |
| Hydroxypropylmethylcellulose K100 | 80 g |
| Lactose | 150 g |
| Stearic Acid | 150 g |
| Carnauba Wax | 180 g |

The above described granulates were transformed into three layer tablets by tableting them in the following order:

| | |
|---|---|
| 1^{st} layer utiiizing granulate 6.1 | 26% |
| 2^{nd} layer utilizing granulate 6.2 | 37% |
| 3^{rd} layer utilizing granulate 6.3 | 37% |

Each tablet had an active ingredient content of 100 mg.

The tablets were analysed after colouring obtaining the following dissolution profile:

Percentage release

| 1 h | 2 h | 4 h | 8 h | 12 h |
|---|---|---|---|---|
| 38 | 55.7 | 75.4 | 90 | 95.5 |

### EXAMPLE 7

Utilizing the granulates previously prepared in example n. 5, two layer tablets containing 150 mg of active ingredient each were prepared.

The two layers had the following composition:

| | |
|---|---|
| granulate 6.2 | 70% |
| granulate 6.3 | 30% |

The so obtained tablets were coloured and checked for the in vitro release obtaining the following results:

Percentage release

| 1 h | 2 h | 4 h | 8 h | 12 h |
|---|---|---|---|---|
| 21 | 47 | 62 | 85 | 100 |

1) Modified release pharmaceutical composition in tablet form containing Bupropion HCl as active substance, characterized in that, as components, hydroxypropylmethylcellulose, stearic acid and carnauba wax mixed with an excipient substance are included.

2) Composition according to claim 1, wherein such excipient substance is lactose.

3) Composition according to claim 1, wherein such components are present in a weight ratio ranging from 0.5 to 2 of Bupropion HCl and the sum of hydroxypropylmethylcellulose, stearic acid as well as carnauba wax.

4) Composition according to claim 1, wherein the weight ratio “hydroxypropylmethylcellulose:stearic acid:carnauba wax” is ranging from “1:1:1” to “1:4:4”.

5) Composition according to claim 2, wherein the weight ratio between Bupropion HCl and lactose is ranging from 1 and 2.

6) Composition according to claim 1, wherein the Bupropion HCl content for each tablet is ranging from 50 mg to 500 mg.
